(19) United States
(12) Patent Application Publication
(10) Pub. No.: US 2026/0364329 A1
(43) Pub. Date: Nov. 19, 2026

(54) Valve Designs to Prevent Air Ingress During Component and Accessory Insertion

(71) Applicant: St. Jude Medical, Cardiology Division, Inc., St. Paul, MN (US)

(72) Inventors: Brian Joseph Perszyk, Shoreview, MN (US); Andrey Kai, Plymouth, MN (US); Christian Elmquist, Mounds View, MN (US)

(73) Assignee: St. Jude Medical, Cardiology Division, Inc., St. Paul, MN (US)

(21) Appl. No.: 19/660,689

(22) Filed: Apr. 28, 2026

Related U.S. Application Data
(60) Provisional application No. 63/808,214, filed on May 19, 2025.

(51) Int. Cl. A61M 39/06

(52) U.S. Cl. CPC A61M 39/0613; A61M 2039/062; A61M 2039/064

(57) ABSTRACT

In some embodiments, a hemostasis valve assembly includes a housing and a valve member positioned within the housing, the valve member including opposed first and second primary surfaces and a valve thickness defined between the first and second primary surfaces, an opening formed in the first primary surface, and a sealing portion disposed about the opening, a well disposed adjacent the sealing portion, first and second slits arranged intersecting each other and extending through a portion of the valve thickness, at least one of the first and second slits being accessible within the opening, and at least one vent hole extending from the first primary surface to the well.

### CROSS-REFERENCE TO RELATED APPLICATIONS

This application claims priority to U.S. Provisional Patent Application No. 63/808,214, filed May 19, 2025, the disclosure of which is hereby incorporated by reference herein.

### BACKGROUND

Sheaths are frequently used to assist in the delivery of medical devices into a patient non-invasively. For example, several types of collapsible and expandable medical devices may be delivered to, and implanted within, the heart of a patient using a sheath that is advanced through the vasculature and into the patient’s heart without needing to make any incisions in the patient’s chest or heart, and without needing to put the patient on cardiopulmonary bypass.

Left atrial appendage (“LAA”) occluder devices are one example of collapsible and expandable medical devices that may be delivered to a patient’s heart via a sheath that traverses the patient’s vasculature. In some examples, a sheath may be advanced through the patient’s femoral vein, into the right atrium through the inferior vena cava, across the atrial septum and into the left atrium, with a distal end of the sheath positioned within or adjacent to the LAA. The LAA occluder device may be within the sheath during the advancement of the sheath, or otherwise may be advanced through the sheath after the sheath is already in the desired position. The LAA occluder may be in a collapsed state with a relatively small profile while inside the sheath, and may self-expand into the LAA upon deployment from the distal end of the sheath. One such LAA occluder is the Amplatzer^{TM} Amulet^{TM} Occluder offered by Abbott Laboratories. One example of a LAA occluder device is described in U.S. Patent No. 10,201,337, the disclosure of which is hereby incorporated by reference herein.

As explained in greater detail below, although this disclosure generally focuses on a steerable sheath for delivering a LAA occluder, the disclosure is not so limited, and may apply to various other types of sheaths (including non-steerable sheaths) and various other types of medical devices to be delivered (including other occluder-type devices, such as PFO closure devices, and other devices that are not occluders).

### BRIEF SUMMARY

In some embodiments, a hemostasis valve assembly includes a housing and a valve member positioned within the housing, the valve member including opposed first and second primary surfaces and a valve thickness defined between the first and second primary surfaces, an opening formed in the first primary surface, and a sealing portion disposed about the opening, a well disposed adjacent the sealing portion, first and second slits arranged intersecting each other and extending through a portion of the valve thickness, at least one of the first and second slits being accessible within the opening, and at least one vent hole extending from the first primary surface to the well.

### BRIEF DESCRIPTION OF THE DRAWINGS

FIG. 1A is a schematic view of a steerable sheath according to one aspect of the disclosure.

FIG. 1B is a schematic representation of another example of a steerable sheath according to one aspect of the disclosure.

FIG. 1C is a schematic cross-sectional view of one example of a steerable sheath according to one aspect of the disclosure.

FIGS. 1D-1M illustrate several examples of deflection of a steerable sheath.

FIG. 1N illustrates examples of different curvatures of a braided transseptal guiding introducer.

FIG. 2 is a schematic view of a dilator for use with the steerable sheath of FIG. 1A.

FIG. 3A is a perspective view of a hemostasis valve assembly of the steerable sheath of FIG. 1A.

FIG. 3B is an exploded view of the hemostasis valve assembly of FIG. 3A.

FIG. 4A is perspective view of the hemostasis valve assembly of FIG. 3A assembled to a hemostasis valve knob.

FIG. 4B is an exploded view of the hemostasis valve assembly and the hemostasis valve knob of FIG. 4A.

FIG. 5A is a perspective view of an occluder in an expanded or deployed condition.

FIG. 5B is a schematic view of the occluder of FIG. 5A in a collapsed or delivery condition.

FIG. 6 is a side view of the occluder of FIG. 5A coupled to a delivery cable.

FIGS. 7A-9A show perspective, rear, side and front views of one example of a valve member 836.

FIGS. 8 and 9A are cross-sectional views taken along cross-section indicators 8-8 and 9-9 of FIG. 7D.

FIG. 9B illustrates advancement of an instrument through the valve member of FIGS. 7A-9A.

FIG. 10 is a schematic cross-sectional view of another example of a valve member having a vent hole.

FIGS. 11A-11E are photographs showing potential locations and shapes of vent holes.

FIG. 12 is a schematic cross-sectional view of another example of a valve member having a vent hole and a flap.

FIG. 13 is a photograph showing fluid purging through the flap.

FIG. 14 is a schematic cross-sectional view of another example of a valve member having no well.

FIG. 15 is a schematic cross-sectional view of another example of a valve member having vent holes.

### DETAILED DESCRIPTION

As used herein, the term proximal refers to a position relatively close to a user of a medical device, while the term distal refers to a position relatively far from the user of the medical device, when the medical device is being used in an intended manner. In other words, the leading end of a medical device is positioned distal to the trailing end of the medical device. As used herein, the term “sheath” is used broadly mean a hollow tube insertable into a patient’s body at an appropriate location for therapeutic treatment and/or diagnostic function(s).

### Delivery Device

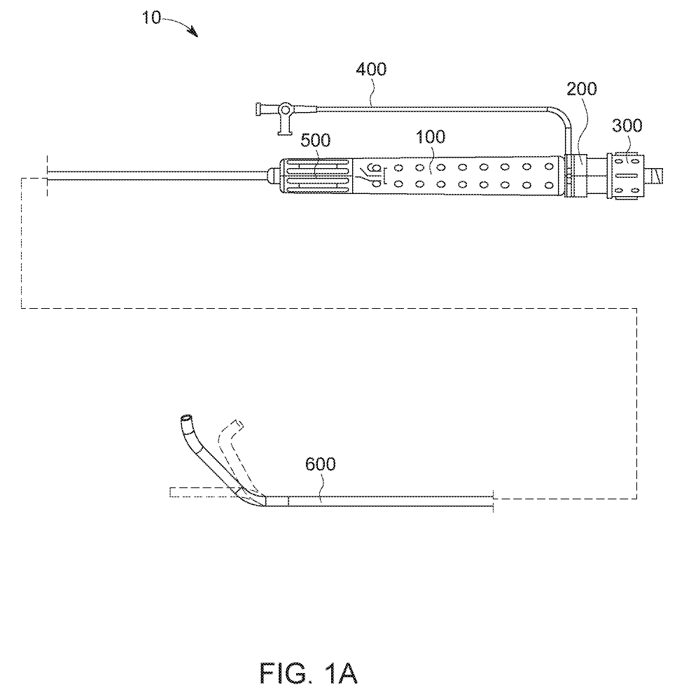

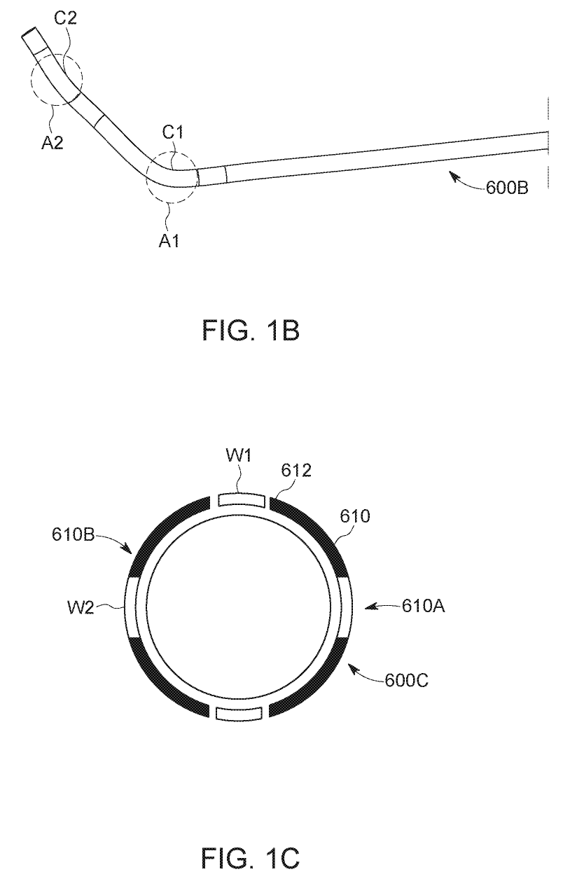

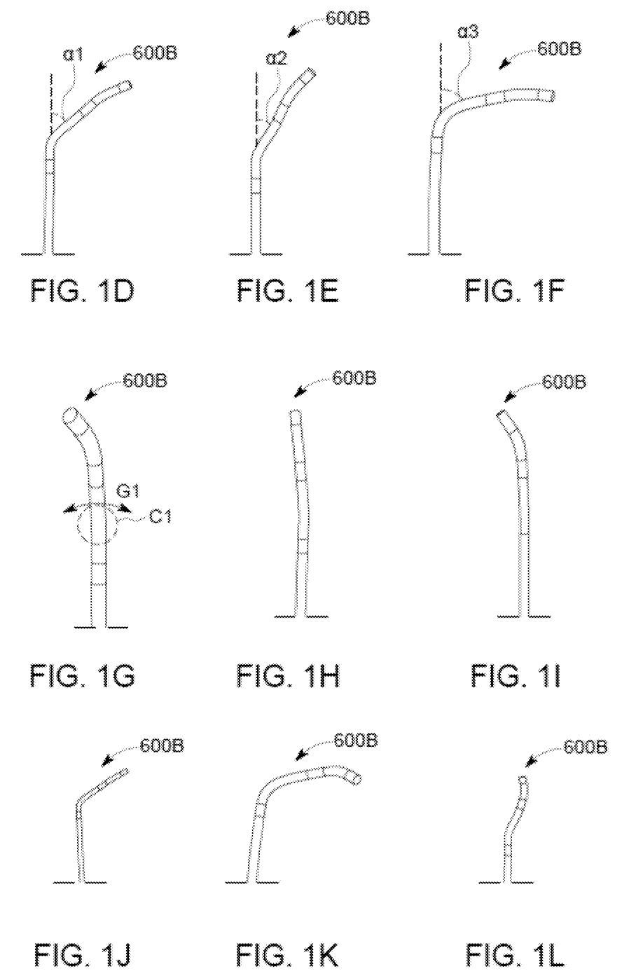

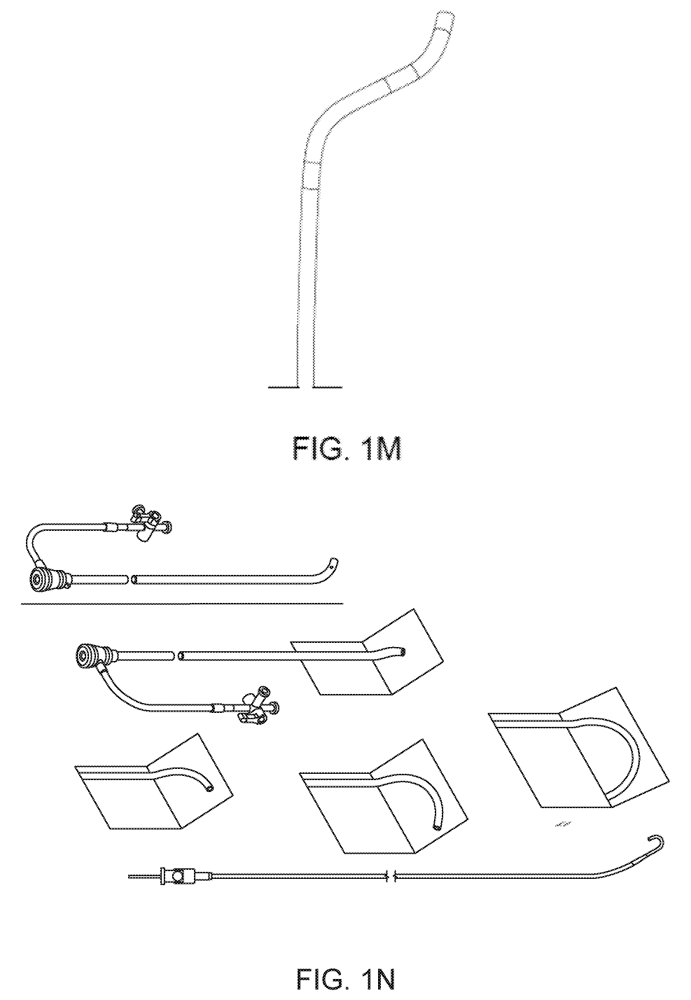

FIG. 1A illustrates a delivery device 10, which in the illustrated embodiment is a steerable sheath, although it should be understood that the inventive concepts disclosed herein may be used in conjunction with other catheter or sheath devices, whether or not steerable. Generally, delivery device 10 includes a handle 100, a hemostasis valve assembly 200 at a proximal end of the handle 100, a hemostasis valve knob (or actuator) 300, a flushing tube 400, a deflection knob 500 at a distal end of the handle 100, and a deflectable sheath 600 extending from a distal end of the deflection knob 500 to a terminal distal end of the delivery device 10. This disclosure includes improvements on various components of delivery device 10, and it will be understood that the features described may be combined in several permutations and that certain features are optional. Delivery device 10 is particularly suited for delivery of a collapsible and expandable LAA occluder, but it should be understood that the delivery device 10 may be suited to delivery of other medical devices.

The handle 100 may be a generally cylindrical or otherwise shaped member that the user of the delivery device 10 may grip during use. The handle 100 may be at least partially hollow and house various components therein, and may have one or more internal lumens so that medical devices may be passed through the delivery device 10 from the proximal end to and beyond the terminal distal end of the delivery device 10. The handle 100 may be rotatably coupled to the deflection knob 500, with the deflection knob 500 being rotatable about the central longitudinal axis of the handle 100. The deflection knob 500 may be operably coupled to two pull wires that traverse the deflectable sheath 600 and which are fixed to anchors (or pull rings or similar structures) near the distal tip of the sheath 600. Rotation of the deflection knob 500 in a first rotational direction may deflect the distal tip of the sheath 600 in a first deflection direction, while rotation of the deflection knob 500 in a second opposite rotational direction may deflect the distal tip of the sheath 600 in a second deflection direction different (e.g., opposite) the first deflection direction. In one exemplary embodiment, the distal tip of the sheath may have a neutral angled position of about 45 degrees relative to the central longitudinal axis of the delivery device 10, with a maximum deflection (upon rotation of the deflection knob 500 in the first, e.g. clockwise, rotational direction) of about 120 degrees or 180 degrees relative to the central longitudinal axis of the delivery device 10, and a minimum deflection (upon rotation of the deflection knob 500 in the second, e.g. counterclockwise, rotational direction) of about 0 degrees (shown in phantom lines in FIG. 1A) relative to the central longitudinal axis of the delivery device 10. In one example, the proximal end of each pull wire may be coupled to an axially slidable component within handle 100, where rotation of the deflection knob 500 causes the two axially slideable components to slide axially in opposite directions. Suitable pull wire mechanisms are described in greater detail in U.S. Patent No. 7,691,095, the disclosure of which is hereby incorporated by reference herein. The deflection mechanisms and ranges described above are merely exemplary, and as noted above, steering or deflection control may in some embodiments be entirely omitted from delivery device 10.

Flushing tube 400 may be a tube with a valve (e.g. luer lock) or connector at a first end thereof, with the second end of the flushing tube 400 (i.e., the end closest to handle 100) being in fluid communication with hemostasis valve assembly 200. The flushing tube 400 may be utilized to introduce fluid into and through the delivery device 10, for example to purge air out of the delivery device 10 prior to use. Flushing tubes are generally well known as they pertain to delivery devices, and thus flushing tube 400 is not described in greater detail herein.

The deflectable sheath 600 may define a lumen therethrough configured to allow other devices to pass through the lumen. The sheath 600 may be formed from any suitable materials and in any suitable configuration. In one example, the sheath 600 includes an innermost liner layer, a torque transfer layer surrounding at least portions of the inner layer, and an outer sheath formed over the torque transfer layer. The wall of the sheath 600 may define lumens as well, for example two lumens spaced about 180 degrees apart, to accommodate the pull wires therethrough. Examples of suitable methods and materials for use in forming the sheath 600 are described in greater detail in U.S. Patent No. 7,914,515, the disclosure of which is hereby incorporated by reference herein.

### Sheath Stiffness

In some examples, sheath 600 may provide stable deployment with near 1:1 torque response. It may be desirable to avoid making the sheath 600 too stiff as doing so can eliminate feedback when contacting cardiac structures and/or reduce the ability of the physician to torque the sheath into position during use. In some examples, sheath 600 may include a torque transfer layer of braided wire, and the stiffness of sheath 600 may be reduced by reducing the braided wire size and/or the extrusion wall thickness of the outer sheath, while maintaining torque strength and kink resistance through braid optimization (e.g., braid wire size, number of wires, higher pick per inch (PPI), braid pattern, etc.). Additionally, reducing the stiffness may also serve to reduce the required forces to deflect one or more of the articulation points.

In some examples, the stiffness of the sheath may be reduced by modifying the braided wire and/or extrusion parameters. In some examples, a sheath may have an extrusion wall thickness of approximately 0.012-0.016 inches, 0.013-0.014 inches, or 0.009-0.012 inches. In some examples, reducing the extrusion wall thickness to 0.009-0.011 inches may reduce the stiffness. In some examples, a braided wire may comprise a 16 wire, 0.003”x0.007” (” represents inches) flat wire braid with a relatively low PPI (e.g., between 30 and 35 picks per inch, or 33 picks per inch as braided prior to removal from the braiding mandrel). Alternatively, a sheath may be formed with a reduced wire size (e.g., 0.0025” x 0.0055”, or 0.002” or 0.003” round wire) to reduce the stiffness. In some examples, changing the braid pattern by increasing the PPI, and/or by moving to a 32-wire diamond pattern with smaller wire (e.g., 0.003” round) may decrease stiffness without affecting torque strength. The number of braids may also be reduced to increase the braid wire angle, which may improve kink resistance for a given stiffness. In some examples, an 8-wire braid at 35-70 PPI may be used.

In some examples, the extrusion durometers for the outer sheath may be modified to reduce the stiffness of the entire sheath, or specific areas of the sheath. For example, to increase flexibility in the proximal curve, which may improve alignment with the LAA, a durometer material below 63D Shore D or according to the Arkema resin standards (e.g., 35D, 40D, 45D, 50D, 55D or a blend thereof) (e.g., Arkema PEBAX® 6333) may be used. Additionally, a more flexible or lower stiffness sheath will reduce the forces required to deflect the sheath, which reduces the pull wire strength requirement, and the torque required for the knob to deflect the sheath. In some other examples, the liner may include HDPE (trilayer) or 72D PEBAX®, to increase the flexibility of the sheath, and improve the durability of the liner. To improve lubricity of the liner (e.g., for device advancement and recapture forces) a lubricant additive like PROPEL+ ®, EVERGLIDE®, and/or MOBILIZE® lubricants may be used.

In some examples, the materials of the sheath may be modified to improve performance. For example, because the distal section needs to articulate separately from the proximal section, it may be softer than the proximal section. The extent to which the distal end is softer may depend on the desired stability of the proximal section during distal deflection, and the amount of curve retention desired during device advancement and deployment. In some examples, the sheath may include 25D PEBAX ® or 35D PEBAX, utilizing 0.002X0.005, 0.0025x.009, or 0.003X0.007 braid. Additionally, the distal section may utilize a laser cut hypotube, as a short section of laser cut hypotube makes manufacturing easier, allows the distal section to flex more easily without kinking or buckling, and may ensure deflection is controlled within a specific plane. After optimizing the distal section for softness, the proximal section may be configured for stability during distal deflection. In some examples, 40D PEBAX ® provides some stability in the proximal section during distal deflection, while 55D PEBAX ® provides even greater stability, but may require more torque to deflect. Thus, in some examples, the proximal section is between 40-55D (or a blend of about 50D) and 0.012-0.016” or 0.009-0.011” wall thickness.

In conjunction with, or instead of the previous configurations, alternate braid wire dimensions may include .0025x.009”, or possibly .0025x.007”. In some examples, pick per inch (PPI) is equal to or less than 28-30 PPI if the braid is transferred onto the mandrel/liner after braiding. It may also be possible to braid directly onto a mandrel, by removing the pull wires from the braid (either during braiding, or after braiding is complete), by pulling out the pull wire, or by cutting the braid that is trapping the pull wire. This eliminates the need to anneal the braid, remove it from the braiding mandrel and transfer it onto the extrusion mandrel. It also allows for a much higher PPI to be braided directly onto the mandrel, which can improve kink resistance and torque strength of the shaft. Ultimately, this method may allow braiding at up 60 PPI.

In some examples, stainless steel wire may be used to form the braid, and several variations are possible. In some examples, braiding directly on the mandrel results in a maximum of approximately 45 PPI-50 PPI for .0025x.0055” braid. In some examples, a coil construction in the distal section may increase flexibility while retaining kink resistance. This may include a flat wire 0.004x.015” (or smaller) with a pitch of 0.030 to 0.060” or a round wire (e.g., 0.002-0.004”) with 0.004 pitch or 0.006-0.30” pitch. Additionally, a smaller wire braid with a coil on top (or underneath) may provide the benefits of a braid (torque strength, resistance to compression and elongation strength), while adding the kink resistance of the coil. One example is a 0.001x0.007” flat wire braid with a 0.002x0.010” coil. A 0.001x.005” flat wire coil and 0.002” and 0.003” round wire coils may also be possible. These may be used with the alternate liner material previously described. Coextrusion may be used in the distal bend area of the distal section to retain flexibility while reducing the extrusion bunching in the outer diameter and reducing the outer surface friction. In some examples, this could include a 25D inner layer with a 35D or stiffer outer layer. In some examples, BaSO4 may be removed in the distal bend area (e.g., in the 25D material) to increase flexibility. A PEBAX® blend may be used in certain areas (e.g., the distal section or the proximal bend area). For example, blends of 20% 45D PEBAX® with 60% 55D PEBAX® and 20% BaSO4, or 20% 55D PEBAX® with 60% 45D PEBAX® and 20% BaSO4 are possible. Blends of 50% 45D, 30% 55D, and 20% BaSO4 are also contemplated.

### Sheath Coatings

In some examples, one or more hydrophilic coatings may be applied to a surface of the sheath to reduce the friction at the sheath outer surface. Such hydrophilic coating(s) may provide the ability for the sheath to be inserted through an 18F introducer (outer) sheath, and may reduce the force of insertion through the groin and across the septum. A low-friction surface may be particularly useful for devices that allow for full recapture without the need for a sheath exchange, and for procedures that contemplate insertion through the groin without an introducer. In some examples, SURMODICS ® coatings (e.g., SERENE^{TM} coatings, PHOTOLINK^{TM} coatings, and/or SERENE® Single-Coat, etc.) may be used.

### Sheath Alignment and Deflection

In some examples, a delivery device may also include features for accommodating an inaccurate transseptal puncture location. For example, when the puncture is located too superiorly, it may be challenging to retroflex off the coumadin ridge to obtain the positioning needed for coaxial alignment. Additionally, for certain anatomies (e.g., a chicken-wing LAA morphology), an out-of-plane distal curvature may be helpful for alignment. To address this, many physicians perform transseptal punctures with a different system (e.g., VERSACROSS® wire, or BRK^{TM} needles), and then exchange it for a steerable sheath/dilator. The added exchange is undesirable as it adds steps and prolongs the procedure. Thus, coaxial alignment may be improved via a more flexible shaft as described above by varying the braid, wire size, pick per inch, braid pattern, extrusion thickness and/or durometer, etc.

Additionally, or alternatively, a sheath 600B may be formed to have two main points of articulation, A1,A2 including a deflectable proximal curve C1 at point of articulation A1 and a deflectable distal curve C2 at point of articulation A2 (See, FIG. 1B). The first point of articulation A1 may be spaced 55-75 mm from the distal tip of sheath 600B, and the second point of articulation A2 may be spaced 10-25mm from the distal tip of sheath 600B, measured from the distal tip to the center of the curve. In some examples, the proximal and/or distal curve plane(s) of deflection may be optimized to accommodate more anatomies. In some examples, the deflectable proximal curve C1 may deflect within the same plane that it is formed in, or it can deflect out of plane, depending on the location of the pull wires. Proximal curve C1 may correspond to one or more additional knobs or steering mechanisms, and may allow the tip of the sheath to deflect in two separate planes, or four completely different directions, depending on where the pull wires are positioned along the circumference of the sheath. In some examples, proximal and distal curves C1,C2 each have two directions of deflection, which results in a four-way deflectable sheath 600B. In some examples, distal curve C2 has two directions of deflection, and proximal curve C1 has four directions of deflection, which results in a six-way deflectable sheath 600B. In some examples, distal curve C2 has four directions of deflection, and proximal curve C1 has two directions of deflection, which results in a six-way deflectable sheath 600B.

The planes of deflection of proximal and distal curves C1,C2 may also be optimized to accommodate more anatomies by changing the position of the pull wires relative to the curve plane that is formed into the sheath. In some examples, one or more pull wire locations are offset to optimize the deflection of the sheath out-of-plane for the most challenging anatomies (e.g., chicken-wing and reverse chicken-wing). In some examples, a four-way deflectable sheath may be formed with a proximal pull ring that is larger than the lumen, liner and pull wires and lumens that run to the distal pull ring. However, this stack-up may result in a thicker wall sheath, or a bump in this area if the extrusions are not modified to accommodate the larger outer diameter at the proximal pull ring. Alternatively, one potential modification is to form a four-way deflectable sheath 600C with a split pull ring 610, having defined channels 612 between the two split halves 610A,610B defining the space where the distal pull wires W1 travel to avoid increasing the outer diameter, the pull wires W1 being orthogonal to proximal pull wires W2 (See, FIG. 1C). It will be understood that other non-orthogonal variations are possible. For example, the proximal pull wires may be disposed anywhere on the split pull ring (i.e., the pull wires do not have to be centered on the split pull ring), such that the proximal and distal pull wires, running in the channels created by the split ring, may be disposed close to or adjacent one another. In some examples, the split rings may be relatively large to maximize the surface area contact for reflow, and ultimately ensure the strength of the pull ring within the liner and outer polymer. The pull ring(s) may have holes in them so that a polymer can flow through the pull ring, locking it into place.

To better illustrate the deflection capabilities of a sheath 600B, a four-way deflectable sheath 600B is shown in various positions in FIGS. 1D-F. It will be understood that the deflection location(s) may be adjusted by varying the location of pull rings, the radius of neutral curvature, and durometers at certain locations. In this example, sheath 600B is shown deflecting to a neutral proximal curvature (FIG. 1D) that forms an angle ɑ1 of approximately 45 degrees with respect to the longitudinal axis of the sheath 600B. As shown in FIG. 1E, sheath 600B may also be capable of superior deflection from the neutral position, the superior deflection forming an angle ɑ2 of approximately 0-45 degrees with respect to the longitudinal axis of sheath 600B. Also, as shown in FIG. 1F, sheath 600B may also be capable of inferior deflection from the neutral position, the inferior deflection resulting in an angle ɑ3 of approximately 45-120 or 45-180 degrees or between 45-90 degrees with respect to the longitudinal axis of sheath 600B.

As shown in FIG. 1G, arrows G1 indicate potential direction(s) for out-of-plane deflection of the proximal curve C1 of sheath 600B. As previously noted, the out-of-plane deflection may be possible at both the proximal curve C1 and/or the distal curve C2. In FIG. 1H, an example of minimal out-of-plane superior deflection of the tip is shown. Conversely, in FIG. 1I, a more out-of-plane superior deflection of the tip is shown. FIGS. 1J-M illustrate additional shapes that can be achieved with sheath 600B, such as a shape having inferior proximal deflection to deflect off the coumadin ridge and superior distal deflection to deflect into a reverse chicken-wing (FIG. 1M).

### Transseptal Punctures

By adding one or more configurations of the articulation described above (e.g., four-way sheath steering or six-way sheath steering), a sheath may provide the ability to achieve a desirable curve shape to more easily perform a transseptal puncture through the steerable sheath. Due to the required length of the proximal section of the sheath, once the sheath crosses the septum to reach the left atrial appendage, it may be desirable to modify the deflection initially, or start with a different neutral position before deflecting to the desired position for left atrial appendage access. In some examples, it may be desirable to maintain a neutral position as-is or in an optimal position for left atrial appendage alignment, and then deflect prior to the transseptal puncture step as needed. Alternatively, it may be desirable to achieve a neutral position optimal for a transseptal puncture. This may include an approach similar to that of a Swartz^{TM} braided transseptal guiding introducer. Examples of different Swartz^{TM} braided transseptal guiding introducer configurations are shown in FIG. 1N. The sheath approach for a transseptal puncture may initially be similar to that of a SL0^{TM} curve, deflect further for transseptal puncture if desired, similar to SL1^{TM} and SL2 ^{TM} curves, and beyond. The sheath may then deflect again for left atrial appendage alignment. In this configuration, one option is to have the neutral curve indicators initially set for left atrial appendage alignment, and for the physician to rotate knobs to the neutral position after a transseptal approach. In some examples, a shortened handle and sheath working length may be desired to accommodate a conventional needle (e.g., a 98 cm BRK^{TM} needle). The handle may be shortened by optimizing and/or reducing the amount of travel required for certain components (e.g., sliding blocks), eliminating dead space in the proximal end of the handle (~.25”), shortening the knobs, shortening the amount of mounting shaft between the knob and the sliding block, replacing the knob mechanism for another (e.g., toggle or slider), shortening the sliding blocks, placing one or more of the sliding blocks at different radial distances or more radially outward, shortening the modified wire guide (e.g., strain relief), shortening the amount of hub extending proximally from the handle, and/or shortening the wire clips that secure the pull wires proximal to the sliding blocks. Additionally, or alternatively, removing the bypass valve, and the external valve housing from the sheath and internalizing it in the handle can reduce some length on the proximal end.

In some examples, instead of articulating the sheath to achieve the optimal starting position for a transseptal puncture, it may be possible to provide a dilator that is sufficiently stiff to force the sheath (at the proximal curve C1, for example) to straighten or partially straighten, and have a soft dilator (and sheath) section just distal to proximal curve C1 that allows flexion in a more typical transseptal curve shape. This may eliminate the need for the physician to articulate the sheath to straighten it out for an initial transseptal puncture shape, only to have to articulate it again for a final transseptal puncture shape. Another dilator option is to decrease the dilator outer diameter in the section of the sheath (and possibly the entire proximal shaft) that requires flexibility during transseptal puncture.

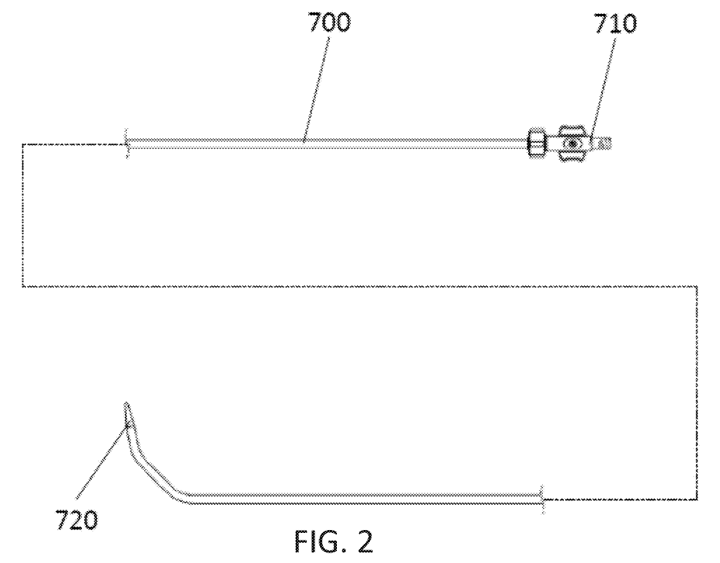

FIG. 2 illustrates a dilator 700 that may be used with delivery device 10. In the illustrated example, dilator 700 is a solid member that includes a connector 710 such as a luer lock at a proximal end thereof, and an atraumatic tip 720 at a distal end thereof. Although referred to as a “solid member,” it should be understood that dilator 700 may include a guidewire lumen passing therethrough to allow for the dilator 700 to ride over a guidewire. In other words, dilator 700 may also be thought of as a “substantially solid” or thick-walled device. The distal end portion of the dilator 700 may, in the absence of applied forces, have a straight orientation, or it may form an angle of about 45 degrees relative to the central longitudinal axis of the dilator 700. In other words, the dilator 700 may include a distal portion that has a neutral angle that is about the same as the neutral angle of the distal tip of the sheath 600, whether that angle is about 45 degrees or another value. The dilator 700 may have an outer diameter that is about equal to (or slightly smaller than) the inner diameter of the sheath 600. Dilator 700 may be positioned within and through the sheath 600 during delivery of the delivery device 10 to the desired anatomical location. Then the dilator 700 may be removed to allow for other devices, such as an LAA occluder, to be passed into and through the delivery device 10 for implantation.

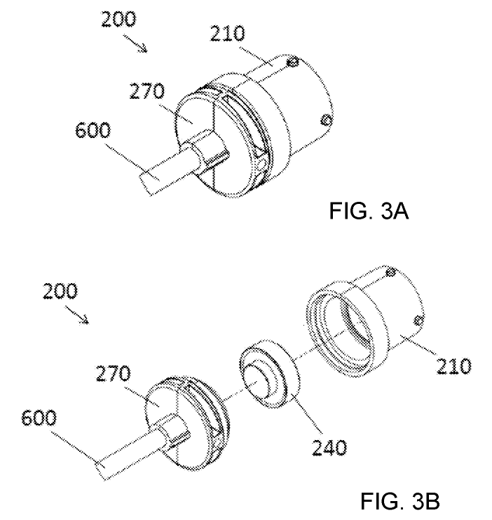

FIG. 3A shows hemostasis valve assembly 200. In the assembled condition, a cap 210 of the hemostasis valve assembly is coupled to a hub 270 of the hemostasis valve assembly, and a proximal end of the sheath 600 is coupled to a distal end of the hub 270. Although not shown in FIG. 3A, the handle 100 may be coupled to and extend from a distal end of the hub 270. FIG. 3B shows hemostasis valve assembly 200 in an exploded view, showing that the hemostasis valve 240 provides a seal between the cap 210 and the hub 270 in the assembled condition of the hemostasis valve assembly 200. Additional features and modifications of the valve will be described in greater detail below.

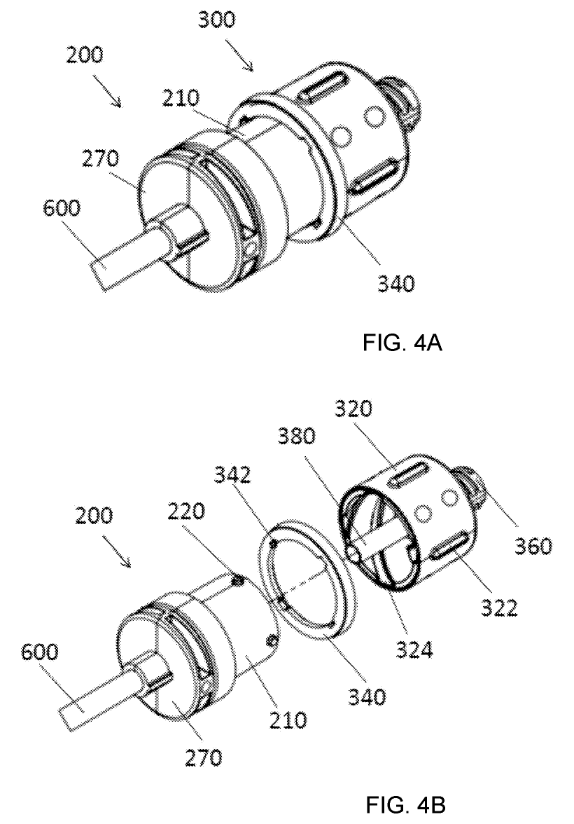

FIG. 4A shows the hemostasis valve assembly 200 assembled to the hemostasis valve knob 300, with FIG. 4B showing a corresponding exploded view. The hemostasis valve knob 300 may include a main body 320, a retaining ring 340, a bypass hub 360, and a bypass tube 380.

Referring generally to FIGS. 4A-B, the main body 320 may be generally cylindrical and may include texturization on an outer surface to enhance a user’s grip on the main body 320. In the illustrated example, the main body 320 includes four raised knurls 322 at equal circumferential spacing to assist a user in torqueing the main body 320. However, it should be understood that other numbers, types, and spacing of texturization features may be provided instead of the raised knurls 322. The main body 320 may have a substantially open distal end, and an inner diameter that is sized to fit over the outer diameter of the main housing 215 of cap 210. As best shown in FIG. 4B, the inner surface of main body 320 may include a plurality of curved channels or recesses 324, for example each in a generally helical configuration. Each curved recess 324 may extend to the terminal distal end of the main body 320, and may have a width and a depth sized to receive a corresponding protrusion 220 therein. With this configuration, when the main body 320 is assembled over the main housing 215, and each protrusion 220 is received within a corresponding curved recess 324, rotating the main body 320 will translate the hemostasis valve knob 300 toward or away from the hub 270 of the hemostasis valve assembly 200

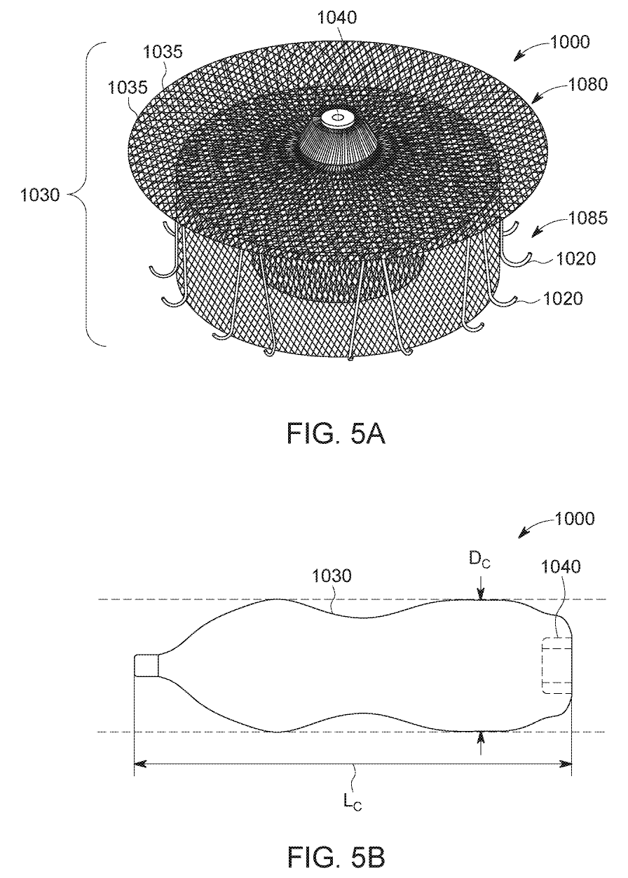

FIG. 5A is a perspective view of an occluder 1000, which may be a LAA occluder. Very generally, occluder 1000 includes an occluding material forming a tubular structure 1030, the occluding material being a braided metal fabric, which may be formed by braiding a plurality of strands 1035, which may be strands of nickel titanium alloy (e.g. under the tradename Nitinol), together and shape set (e.g. via heat setting) to the shape shown in FIG. 5A. When in the shape-set or expanded condition (e.g. in the absence of applied forces), the occluder 1000 may include a generally disk-shaped portion 1080 configured to abut an ostium of the patient’s LAA, and a second generally cylindrical portion 1085 configured to be received within the patient’s LAA. In some embodiments, the disk-shaped portion 1080 may be coupled to the cylindrical portion 1085 via a small diameter transition portion. The cylindrical portion 1085 may include one or more hooks 1020 configured to engage tissue within the LAA upon deployment. The disk-shaped portion 1080 may include a connector 1040, which may for example include internal threads, for coupling to a delivery cable 1300.

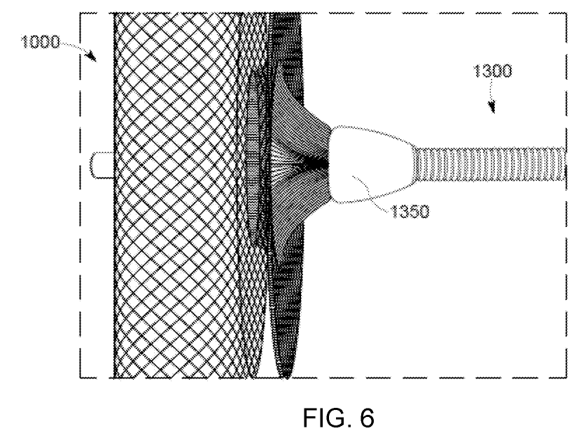

FIG. 5B illustrates occluder 1000 in a collapsed condition, having a collapsed diameter D_{C} smaller than the diameter of the disk-shaped portion 1080 and the cylindrical portion 1085 when in the expanded condition shown in FIG. 5A, and a collapsed length L_{C} longer than the length of the occluder in the expanded condition shown in FIG. 5A. FIG. 6A illustrates the delivery cable 1300 coupled to the connector 1040, for example via a threaded hub 1350 of the delivery cable 1300 that has been threaded into the connector 1040.

An exemplary use of the delivery device 10 for delivering occluder 1000 is described below. At the beginning of the procedure (or in preparation thereof), the delivery device 10, including a dilator, may be removed from sterile packaging. The user preferably confirms that the hemostasis valve knob 300 is in the first, sealed position such that the hemostasis valve 240 is closed. If the hemostasis valve knob needs to be adjusted, the user may rotate the hemostasis valve knob 300, for example by turning it counter-clockwise relative to the hemostasis valve assembly 200, to retract the bypass tube 380 so that it does not pass through the hemostasis valve 240. Alternatively, an alternative valve configuration such as those disclosed herein, including a passive hemostasis valve assembly may be used, which does not require adjustment. The sheath 600 and the dilator 700 may be wiped with sterile gauze dampened with sterile saline to remove any foreign material that may be on the components. The user may then pass the dilator 700 through the delivery device 10 until the distal end of the dilator 700 passes the distal end of the sheath 600. Even though the hemostasis valve knob 300 is in the first, sealed position, the dilator 700 has enough column strength to readily pass through the hemostasis valve 240 without assistance of the bypass tube 380, without any buckling or damage occurring to the dilator 700. When the dilator 700 is fully inserted through the delivery device 10, the connector 710 (or a threaded collar associated with the connector 710) may be rotated to couple to the bypass hub 360, for example by inner threads of the connector 710 engaging outer threads 362 of the bypass hub. The dilator 700 is now coupled to the remaining portions of the delivery device 10 as a single unit. Access may be gained to the patient via any suitable method, and a guidewire may be advanced into the patient’s vasculature until it reaches the patient’s left atrium, LAA, or pulmonary vein. Prior to or during introduction of the guidewire, a puncture may be made through the patient’s atrial septum if the delivery route is, for example, through the patient’s femoral vein and to the right atrium via the inferior vena cava, with the septal puncture allowing the guidewire and other components to traverse the atrial septum into the left atrium. With the guidewire in place, the dilator 700 (and the remainder of the delivery device 10 to which it is coupled) may be advanced over the guidewire into the patient until the distal end of the delivery device 10 reaches the left atrium or LAA. With the distal end of the delivery device 10 in the desired location, the connector 710 may be rotated to decouple it from the bypass hub 360, and the dilator 700 may be withdrawn from the sheath 600, preferably slowly to prevent any ingress of air. After the dilator 700 is fully removed, the guidewire may next be fully removed from the delivery device 10 and the patient, although the guidewire may instead be simultaneously removed with the dilator 700. With the distal end of the sheath 600 in the desired position, the occluder 1000 may be introduced into and through the delivery device 10.

### Valve Modifications

As briefly noted, valves are commonly utilized on the proximal end of delivery sheaths to provide hemostasis and prevent air ingress throughout the procedure. Various types of valves are known, with cross slit valves being commonly used. Cross slit valves can seal on a variety of instrument diameters effectively. Circumferential seals formed of sealing portions may be added to cross slit valves to improve sealing. While circumferential seals do not provide hemostasis on their own, they are effective at providing hemostasis on larger diameter shafts or accessories that are inserted therethrough. Therefore, the combination of a cross slit valve and a circumferential seal with radially-extending sealing portions may be useful.

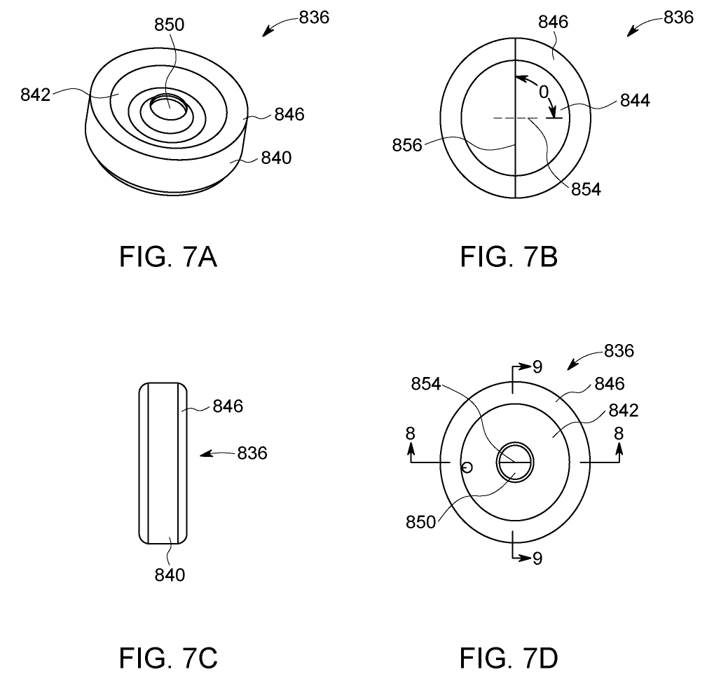

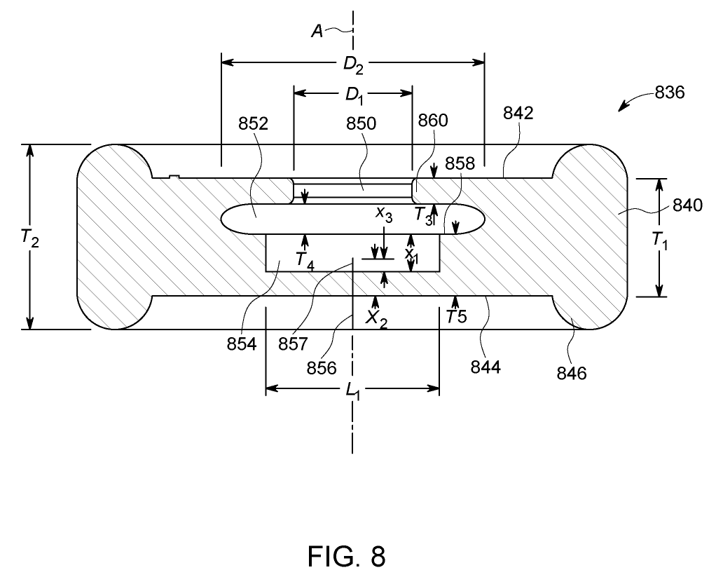

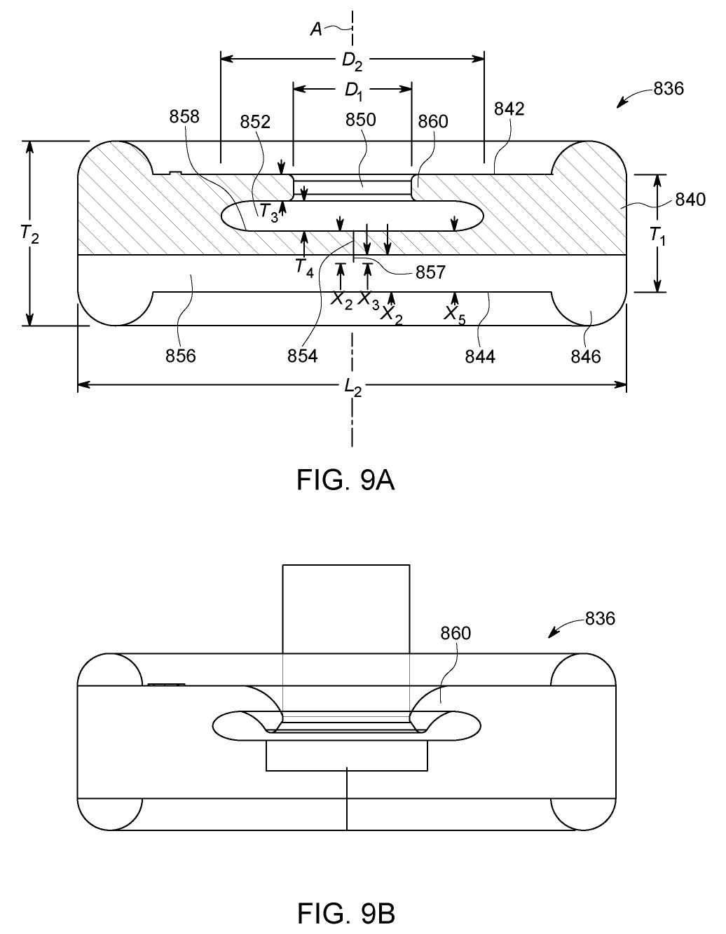

Referring now to FIGS. 7A-9A, one example of such a valve, valve member 836, is shown and described in further detail. FIGS. 7A-7D show perspective, rear, side and front views, respectively, of valve member 836. FIGS. 8 and 9A are cross-sectional views taken along cross-section indicators 8-8 and 9-9 of FIG. 7D. FIGS. 8 and 9A also show valve member 836 having perimeter surface 840, first and second primary surfaces 842, 844 arranged opposite of each other, and rim 846 extending around the perimeter surface 840. Valve member 836 also includes opening 850, well 852, and first and second slits 854, 856. Second slit 856 may fully or partially extend from one side of rim 846 to the other. First and second slits 854, 856 may include overlapping portion 857. First slit 854 may be formed in and accessible on bottom surface 858 of well 852. Sealing portion 860 may be positioned between opening 850 and well 852. Rim 846 may extend from at least one of first and second primary surfaces 842, 844. In the depicted embodiment, rim 846 extends axially away from both first and second primary surfaces 842, 844. Rim 846 may help maintain a position of valve member 836 relative to the housing and cap.

Valve member 836 may have a thickness T1 measured between first and second primary surfaces 842, 844, as shown in FIG. 8. Rim 846 may have a thickness T2 as shown in FIG. 8. In one example, thickness T1 may be in the range of about 0.1 inch (2.54 mm) to about 0.2 inch (5.08 mm), and more particularly about 0.16 inch (4.06 mm) to about 0.19 inch (4.82 mm). Thickness T2 may be in the range of about 0.2 inch (5.08 mm) to about 0.35 inch (8.89 mm), and more particularly about 0.25 inch (6.35 mm) to about 0.3 inch (7.62 mm). Thickness T1 is typically dependent at least in part on the materials of valve member 836. In one example, valve member 836 comprises silicone material. The silicone material may have a durometer in the range of, for example, about 10 durometer to about 50 durometer, and more particularly about 20 durometer to about 40 durometer. Other materials for use in valve member include, for example, low durometer urethane and low durometer rubber.

Thickness T1 is selected to optimize leak integrity while minimizing the amount of insertion force required to pass a medical instrument through valve member 836. Typically, the smaller the thickness T1 the less leak integrity is provided and the least amount of insertion force is required to pass a medical instrument through valve member 836. The greater the thickness T1 the greater the leak integrity and the greater amount of force is required for insertion of the medical instrument. Further, providing an increased thickness T1 reduces the possibility of tearing or causing other damage to valve member 836 as a result of repeated use or from insertion of relatively large diameter medical instruments.

Opening 850 is formed in first primary surface 842. Opening 850 has a diameter D1. Opening 850 has a thickness T3 measured in a direction from first primary surface 842 toward second primary surface 844. Opening 850 extends coaxially with a central axis A of valve member 836. Opening 850 may have a circular shape. Other shapes are such as an oval shape are possible for opening 850.

The diameter D1 of opening 850 may vary depending on the size of a medical instrument expected to pass through opening 850. Typically, diameter D1 is slightly smaller than the smallest large diameter medical instrument extending through opening 850. The material composition of valve member 836 may permit enlarging of opening 850 as the medical instrument is inserted there through. For example, diameter D1 may increase in size in the range of about 10% to about 100%, and more particularly in the range of about 10% to about 50% in size from a rest state in which no expansion force is applied to opening 850 to an expanded state caused by insertion of the medical instrument.

Well 852 is positioned between opening 850 and first and second slits 854, 856. Well 852 may be positioned in series with opening 850 between first primary surface 842 and second primary surface 844. Well 852 may be referred to as a recess, chamber, or cavity. Well 852 may be formed coaxially with opening 850 and central axis A. Well 852 may have a circular shape. Well 852 may have the same shape as opening 850. Well 852 has a diameter D2. Diameter D2 is typically greater than D1. Well 852 may extend through thickness T4 between opening 850 and first slit 854. Well 852 may provide a space or cavity within which sealing portion 860 moves as a medical instrument passes through and deforms opening 850. The relatively low durometer, elastic properties of the material of valve member 836 permits some expansion, deformation and/or elastic stretching of sealing portion 860 surrounding opening 850 as sealing portion 860 moves into well 852. The difference in diameter D1 and D2 along with the thickness T4 define at least in part the amount of space available for sealing portion 860 to move into.

Diameter D2 may be relatively constant for a range of diameters D1. For example, diameter D2 may be in the range of about 0.35 inch (8.89 mm) to about 0.5 inch (12.7 mm), and more particularly in the range of about 0.4 inch (10.16 mm) to about 0.43 inch (10.92 mm). Diameter D1 may be in the range of about 0.1 inch (2.54 mm) to about 0.19 inch (4.82 mm) for any given size of D2. Alternatively, the diameter D1 may be proportional to diameter D2 and maintain the proportionality for any given size of opening D1. For example, D1 may be in the range of about 30% to 60% of D2, such as about 50% of D2.

Opening 850 and associated sealing portion 860 may provide a first sealing point between valve member 836 and a medical instrument extending through valve member 836. The seal provided at the first sealing point may be a fluid-tight seal that limits flow of liquids or gases between valve member 836 and the medical instrument passing there through.

First and second slits 854, 856 may be arranged in series between first and second primary surfaces 842, 844. First slit 854 may be formed in bottom surface 858 of well 852 and may extend to a depth X1 and have a length of L1. Second slit 856 may extend from second primary surface 844 towards first slit 854 to a depth X2 and have a length of L2. In some examples, length L2 may encompass the entire length from one end of rim 830 to an opposing end. The depths X1 and X2 may be substantially equal.

First and second slits 854, 856 may have linear shapes along their lengths L1, L2. Other shapes such as contoured shapes along lengths L1, L2 are also possible. Portions of first and second slits 854, 856 may overlap in the axial direction along central axis A to provide overlapping portion 857. Overlapping portion 857 may be oriented at an intersection point between first and second slits 854, 856 and may also be referred to as intersection point 857 or intersection 857. Overlapping portion 857 has a depth X3 that is usually less than X1 and X2.

First and second slits 854, 856 may provide a pathway or a channel that extends from bottom surface 858 to second primary surface 844. The portion of valve member 836 through which first and second slits 854, 856 extend may have a thickness T5. The thickness T5 may be in the range of, for example, about 0.08 inch (2.03 mm) to about 0.1 inch (2.54 mm), and more particularly in the range of about 0.85 inch (21.6 mm) to about 0.95 inch (24.13 mm).

The depths X1 and X2 may be in the range of, for example, about 0.3 inch (7.62 mm) to about 0.6 inch (15.24 mm), and more particularly in the range of about 0.4 inch (10.16 mm) to about 0.5 inch (12.7 mm). Depth X3 of overlapping portion 857 may be in the range of, for example, about 0.01 inch (0.254 mm) to about 0.1 inch (2.54 mm), and more particularly in the range of about 0.015 inch (0.381 mm) to about 0.03 inch (0.762 mm).

The lengths L1, L2 may vary depending on the various conditions and features of valve member 836. For example, L1 may be limited by the diameter D2 of well 852. Typically, diameter D1 of first slit 854 is less than diameter D2. First slit 854 may be formed by inserting an instrument such as cutting device through opening 850 and well 852 and into contact with bottom surface 858.

Second slit 856 is formed in second primary surface 844. Length L2 of second slit 856 may have fewer constraints. Length L2 may be as great as a maximum diameter of valve member 836, as shown in the figures. In other arrangements, length L2 is equal to or less than the length L1. In one example, length L1 is in the range of about 0.25 inch (6.35 mm) to about 0.3 inch (7.62 mm), and more preferably about 0.27 inch (6.86 mm) to about 0.28 inch (7.11 mm). Length L2 may be in the range of, for example, about 0.8 inch (20.32 mm) to about 0.95 inch (24.13 mm), and more particularly in the range of about 0.85 inch (21.59 mm) to about 0.89 inch (22.61 mm).

First and second slits 854, 856 may be arranged at an angle θ relative to each other as shown in FIG. 7B. The angle θ may be in the range of about, for example, about 45° to about 90°. First and second slits 854, 856 may be arranged generally perpendicularly to each other and may be referred to as intersecting slits. While two slits 854, 856 are shown, other slit arrangements are possible including a single slit, or three or more slits.

Turning back to FIG. 8, when a sealing portion 860 is used with a first slit 854 of a cross-slit seal, well 852, formed between the two, may be introduced to allow the function and/or movement of the sealing portion 860, independent of first slit 854. In this configuration, if sealing portion 860 seals against the inserted shaft before the shaft breaches the first slit 854, the air that is trapped between the two elements can be forced through the first slit 854 as it is being breached. FIG. 9B illustrates an instance in which an instrument (e.g., a sheath) is advanced through valve member 836, deforming sealing portion 860 distally, which creates a sealed pocket potentially full of air. Without being bound by any particular theory, it is believed that this leak is more likely to occur with shafts that have a blunt end such as an LAA occluder loader, than a shaft with a tapered end like a dilator. Air entering the sheath is undesirable because there is a potential for air embolism that can lead to patient harm.

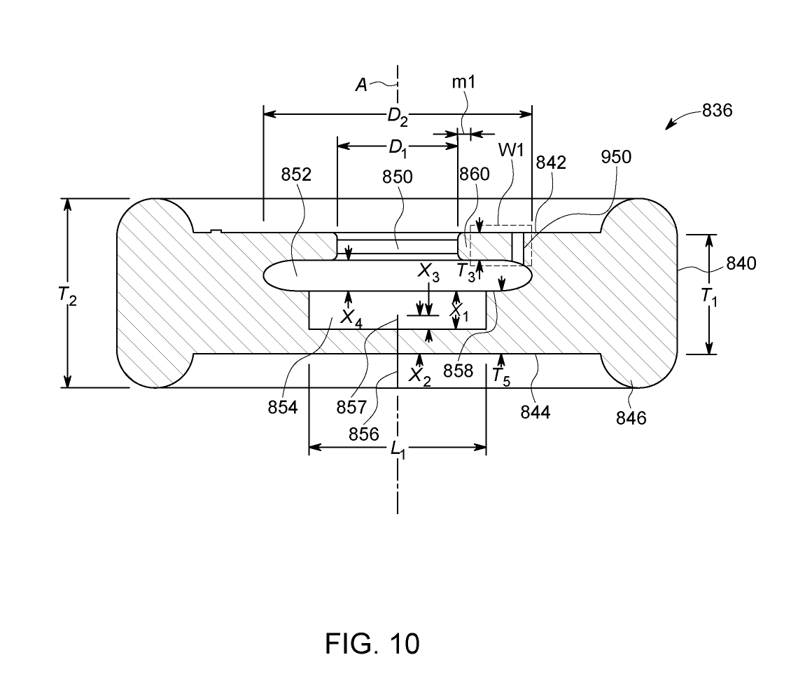

As shown in FIG. 10, valve member 836 may be further modified to provide methods of evacuating air. Specifically, one or more vent holes 950 may be added to allow the air within well 852 between sealing portion 860 and first slit 854 to evacuate before the shaft or other instrument breaches the first slit 854. Vent hole 950 may be formed as a generally longitudinal passageway, lumen or bore that connects well 852 to the outside of valve member 836 (i.e., through first primary surface 842). Vent hole 950 may be perpendicular to a longitudinal axis of well 852. For the vent hole 950 to properly function, the shaft (e.g., an LAA occluder loader) may be flushed while it is being inserted through the valve. The fluid exiting the shaft/loader may purge the empty space of air, minimizing or eliminating the air from the space between the seals as the shaft is advanced. Then, as the shaft enters, it flexes the sealing portion 860. When the seal flexes, preferably the vent holes close, but if the vent holes remain open the amount of leak through the sealing portion 860 will be limited by the size of the vent holes (i.e. the smaller the vent holes, the more the wiper seal will resist leak). In some examples, it may be useful to slow the flush to ensure that if air does enter the sheath, it only enters the valve area and is not forced down the sheath.

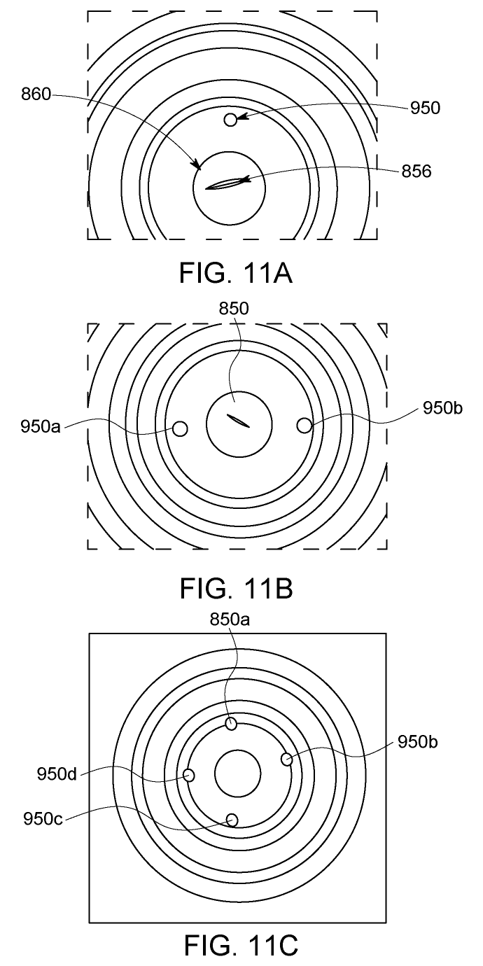

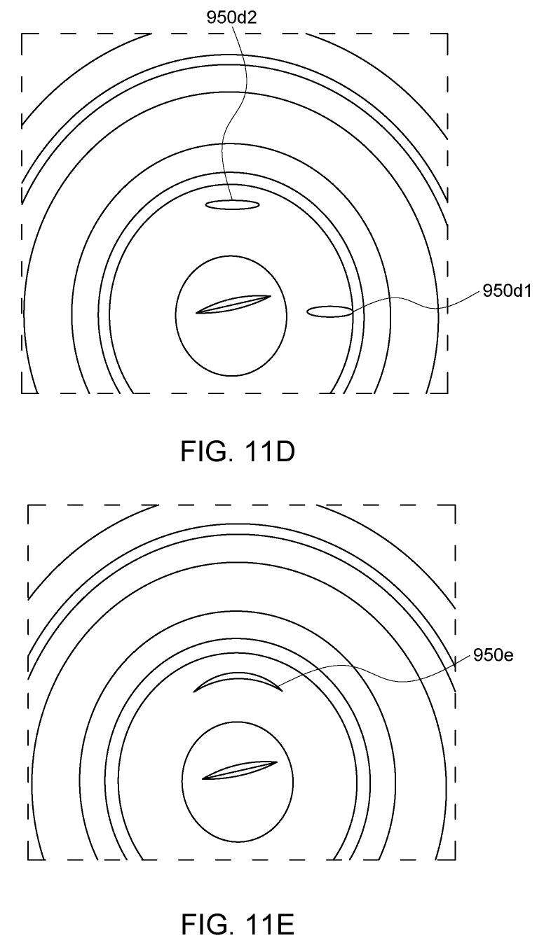

A valve member 836 with a single vent hole 950 is shown in FIG. 11A and it is formed across sealing portion 860. It will be understood that multiple vent holes may be formed. For example, FIG. 11B shows two vent holes 950a,950b that are diametrically opposed on two sides of opening 850. FIG. 11C shows four vent holes 950a-950d that are disposed on four sides of opening 850 at the 12 o’clock, 3 o’clock, 6 o’clock and 9 o’clock positions. Vent holes 950a-950d may be circumferentially equidistant from one another. Vent hole(s) 950 may vary in diameter from approximately 0.005” to approximately 0.040”. If multiple vent holes 950 are used, they may all be of a same diameter or of different diameters. Moreover, the number of vent holes may be determined by the effectiveness of the purge and the size of the vent holes (i.e., smaller vent holes may require more vent holes for an effective purge, but may provide more resistance to leak). The position of the vent hole(s) 950 may vary with respect to the sealing portion 860 anywhere within window w1. In some examples, the position of vent holes 950 may be at the maximum distance from the opening (i.e., at the perimeter of well 852, as shown in FIG. 10). In some examples, the minimum distance m1 from opening 850 may occur at approximately 0.035”, to ensure maintained functionality with the desired mating shafts. This minimum is intended to ensure that not all the vent holes are covered when the loader tube contacts the circumferential seal. Moving the vent holes further away from the center of the valve may be desirable, to prevent accessories, such as the dilator, from catching on the vent hole during insertion of the accessories.

Additionally, any of the vent holes may be oval or ellipse-shaped. As shown in FIG. 11D, vent holes 950d1,950d2 may be disposed about the opening, and the vent hole orientation may be radially aligned with the valve radial direction such as vent hole 950d1 or perpendicular to the valve radial direction as shown with vent hole 950d2. Alternatively, as shown in FIG. 11E, a vent hole 950e may be curved and shaped so that the inner and outer edges are concentric with the valve, which may promote better closure of the vent hole(s) when accessories are advanced therethrough. Optionally, the ends of the oval vent hole(s) may also be narrowed to further promote closure. These holes may be between .003” and .015” in width, and between 0.030” and .250” in length.

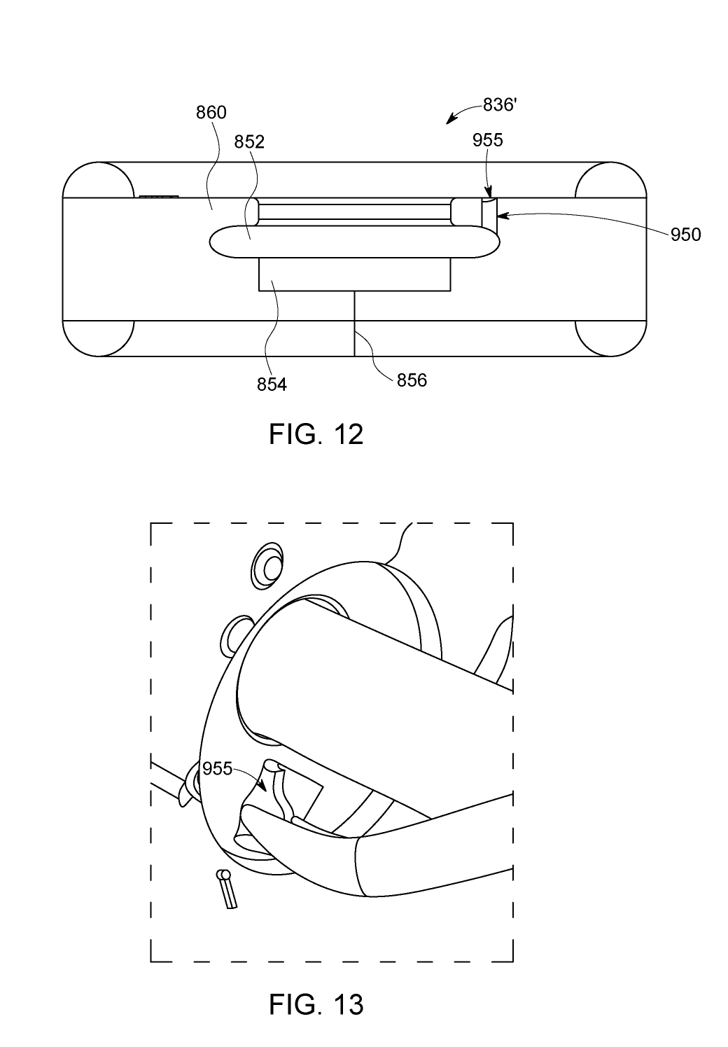

In some examples, in addition to the vent holes, flaps on the proximal side of the circumferential sealing portion 860 may be added to improve sealing from air ingress. FIG. 12 shows a valve member 836’ with vent hole 950 and an added flap 955 to improve sealing from air ingress. In this example, flap 955 acts as a check valve that allows air to purge from the space between the two seals, but closes if a vacuum is formed inside of the sheath (e.g., if a patient snores when they are under conscious sedation). A single flap 955 may be added to cover each vent hole and multiple flaps 955 may be provided for multiple vent holes. In some examples, flap 955 may be created by cutting into the valve material, lifting it out of the way, and punching the hole. Alternatively, flap 955 may be formed as a separate thin piece of polymeric material that is adhered to the valve. It will be understood that the size, number, and/or position of the vent holes may vary and that the size, number, and/or position of each flap may correspond to that of the vent holes. FIG. 13 is a photograph showing a vent with an adhered flap 955 and fluid being purged during loader insertion.

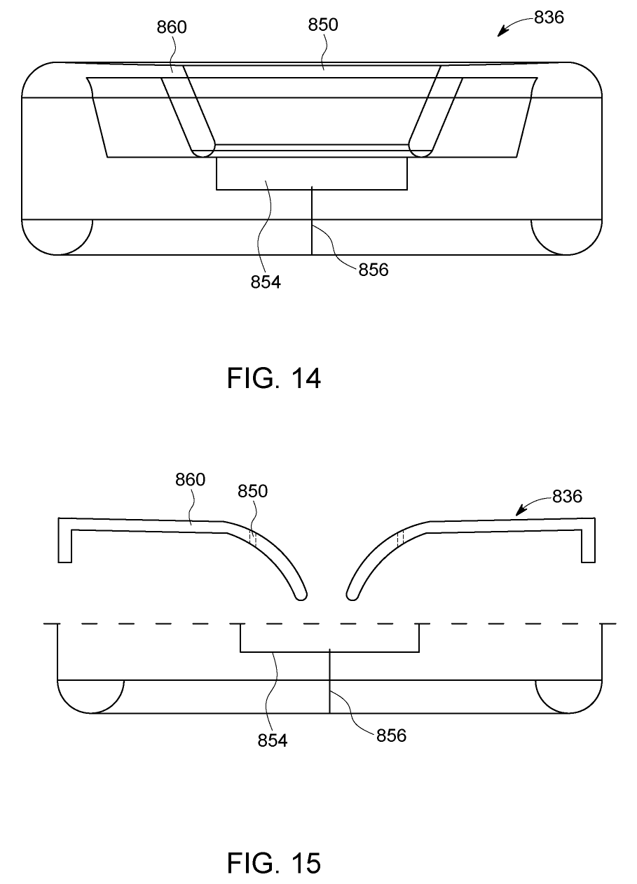

FIG. 14 illustrates yet another embodiment, in which valve member 836” has been modified to eliminate the well between sealing portion 860 and first slit 854 and eliminate the need for venting. In this example, a two-part seal may be formed where the cross-slit seal and circumferential seal are molded separately and positioned relative to one another within a valve housing. Valve member 836” may include opening 850 and all the other components previously described with reference to FIGS. 7A-9, but notably sealing portion 860 is angled and no well is formed between sealing portion 860 and first slit 854.

According to another example, FIG. 15 illustrates a modified valve 836’’’ with sealing portion 860 that has incorporated vent holes 950. In this configuration, the vent holes 950 may allow purging of the air at initial contact, but upon further advancement of the accessory, the vent holes 950 may become occluded by the accessory. In some examples, vent holes 950 may be positioned just beyond (radially) the largest accessory that needs to advance through the valve (e.g., an LAA occluder loader) or approximately 0.110” from the center of the opening. Alternatively, the vent hole 950 may be cut into the seal parallel, or substantially parallel to the axis of the valve, so that the vent hole 950 may also seal due to compression of the material. The vent holes may also be a shape other than a circle, as described above. In this embodiment, if the sealing portion 860 is separated from the cross slit seal, it may be made from a softer material than the main seal, such that it more easily flexes and compresses for improved vent hole closure. In this example, the sealing portion 860 may be positioned immediately adjacent to the cross slit seal, or it may be separated by a well. If the two seals are separately formed, they may be secured together within a valve housing (not shown).

Although the features (e.g., the hemostasis valves, the deflecting curves, the handle configurations, etc.) described herein may be particularly useful for the LAA occluder 1000 device described herein, it should be understood that this particular use is merely exemplary. For example, any of the features may be used in combination with delivery systems having any one of the valves (or no valve), or having steerable or fixed sheaths. Additionally, any of the features described herein may be particularly useful for other occluders formed of braided metal, including septal occluders, patent ductus arteriosus (“PDA”) occluder devices, patent foramen ovale (“PFO”) closure devices, paravalvular leak closure devices, etc. It should further be understood that the features described herein may work well with other occluders, including those not formed from a braided mesh, or any other medical device, whether an occluder or not and whether formed of braided mesh or not, if that medical device has relatively low column strength and/or if it would be undesirable for that medical device to be contaminated with silicone oil or another lubricant from direct contact with a hemostasis valve. Additionally, the disclosed features may be formed with others in various combinations.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A hemostasis valve assembly, comprising:
a housing; and
a valve member positioned within the housing, the valve member comprising:
opposed first and second primary surfaces and a valve thickness defined between the first and second primary surfaces;
an opening formed in the first primary surface;
a sealing portion disposed about the opening;
a well disposed adjacent the sealing portion;
first and second slits arranged intersecting each other and extending through a portion of the valve thickness, at least one of the first and second slits being accessible within the opening; and
at least one vent hole extending from the first primary surface to the well.

2. The hemostasis valve assembly of claim 1, wherein the at least one vent hole extends through the sealing portion.

3. The hemostasis valve assembly of claim 1, wherein the at least one vent hole is perpendicular to a longitudinal axis of the well.

4. The hemostasis valve assembly of claim 1, wherein the at least one vent hole comprises a single vent hole.

5. The hemostasis valve assembly of claim 1, wherein the at least one vent hole comprises multiple vent holes spaced from one another.

6. The hemostasis valve assembly of claim 5, wherein the multiple vent holes are circumferentially disposed about the opening.

7. The hemostasis valve assembly of claim 1, wherein the at least one vent hole is between 0.005” and 0.040” in diameter.

8. The hemostasis valve assembly of claim 1, wherein the opening has a first diameter and the well has a second diameter, the first diameter being smaller than the second diameter.

9. The hemostasis valve assembly of claim 1, wherein the opening and well are arranged coaxially with an intersection point between the first and second slits.

10. The hemostasis valve assembly of claim 1, wherein a size of the opening is expandable.

11. The hemostasis valve assembly of claim 1, wherein the first and second slits are arranged perpendicular to each other.

12. The hemostasis valve assembly of claim 1, wherein the second slit has a greater length than the first slit.

13. The hemostasis valve assembly of claim 1, further comprising at least one flap, each of the at least one flap covering a corresponding one of the at least one vent hole.

14. The hemostasis valve assembly of claim 13, wherein the at least one flap is unitarily formed with material of the sealing portion.

15. The hemostasis valve assembly of claim 13, wherein the at least one flap is separately formed and coupled to the sealing portion.

16. A valve member, comprising:
opposed first and second primary surfaces and a valve thickness defined between the first and second primary surfaces;
an opening formed in the first primary surface;
a sealing portion disposed about the opening;
first and second slits arranged intersecting each other and extending through a portion of the valve thickness, at least one of the first and second slits being accessible within the opening and being disposed directly adjacent the sealing portion; and
at least one vent hole extending through the sealing portion.

17. The valve member of claim 16, further comprising a well disposed adjacent the sealing portion, the sealing portion being sandwiched between the well and the primary surface.

18. The valve member of claim 16, wherein the at least one vent hole is perpendicular to a longitudinal axis of the well.

19. The valve member of claim 16, wherein the at least one vent hole comprises a single vent hole.

20. The valve member of claim 16, wherein the at least one vent hole comprises multiple vent holes spaced from one another.
